(19) United States
(12) Patent Application Publication
(10) Pub. No.: US 2019/0231255 A1
(43) Pub. Date: Aug. 1, 2019

(54) SYSTEM WITH VITAL DATA SENSOR

(71) Applicant: Vorwerk & Co. Interholding GmbH, Wuppertal (DE)

(72) Inventors: Andrej Mosebach, Bochum (DE); Christian Holz, Dortmund (DE)

(21) Appl. No.: 16/263,935

(22) Filed: Jan. 31, 2019

(30) Foreign Application Priority Data
Feb. 1, 2018 (EP) 18154689.6

(51) Int. Cl. A61B 5/00; G06N 20/00; A61B 5/053; H04L 12/28; A47L 9/28

(52) U.S. Cl. CPC A61B 5/4809; A61B 5/7282; G06N 20/00; A47L 9/2805; A61B 5/7225; H04L 12/2821; A61B 5/0531

(57) ABSTRACT

The present disclosure concerns a system comprising an evaluation unit and a sensor, wherein the sensor can measure a vital parameter of an organism and the evaluation unit allows to conduct an evaluation based on the measured vital parameter. The evaluation unit is configured such that the evaluation unit can send a command signal to an external device, particularly to a household appliance, in dependency of a result of the evaluation, so that the external device carries out an action based on the command signal. The present disclosure also concerns a use, a method and a computer program product. The user can thereby save time and a very high operating comfort can be realized.

### PRIORITY CLAIM

This application claims priority to European Application No. 18154689.6, filed Feb. 1, 2018, which application is hereby incorporated in its entirety herein.

### FIELD OF THE DISCLOSURE

The present disclosure concerns a system with an evaluation unit and a sensor. The sensor can measure a vital parameter of an organism. The evaluation unit allows to conduct an evaluation based on the measured vital parameter.

### BACKGROUND

Vital parameters such as blood pressure provide information about the current condition of an organism. So-called fitness trackers record a vital parameter and thus give an athlete, for example, feedback on his physical condition. Examples of more of such systems are described in the documents EP0496196A1, WO2017/037250A1, US2016/100696A1 and US2015/094544A1.

It is object of the present disclosure to provide a further developed system that enables an enhanced functionality in connection with other devices.

### SUMMARY

For solving the problem, a system according to the main claim and a use, a method as well as a computer program product according to the other independent claims are provided. Preferable embodiments are described in the dependent claims.

In order to solve the problem, a system comprising an evaluation unit and a sensor is provided. The sensor can measure a vital parameter of an organism. The evaluation unit allows to conduct an evaluation based on the measured vital parameter. The evaluation unit is configured such that the evaluation unit can send a command signal to an external device, particularly to a household appliance, in dependency of a result of the evaluation, so that the external device carries out an action based on the command signal. The user can thereby save time and a very high operating comfort can be realized.

A vital parameter refers to an organism (living being) and can be specified by a measured value. A vital parameter usually describes a basic function or vital function of the organism.

The vital parameter, i.e. the measured value, can be determined using the sensor. Examples of vital parameters are body temperature, heart rate, respiratory rate or blood pressure. In particular, the organism is a human being.

The system, which allows an external device to carry out an action based on a measured vital parameter, enables an enhanced functionality in connection with external devices, which is explained below using a some examples.

The external device can be a front door lock, a roller shutter and/or smart home server. In one embodiment, the action-triggering result is a fall-asleep-event of the organism. The external device can thus carry out an action or take a certain operating state immediately after the fall-asleep-event, i.e. the point in time of falling asleep. For example, the front door lock can be locked automatically, roller shutters and/or windows can be closed automatically. In one embodiment, the action-triggering result is a wake-up-event of the organism. For example, the heating, especially in the bathroom, can then be activated immediately after the organism wakes up, so that the organism, for example a person, can enter a preheated bathroom after waking up.

The organism can be an animal or a pet. The owner of the animal or pet can be notified immediately after the animal or pet wakes up, for example to close doors and windows. Alternatively or additionally, the smart home server can be the external device, which is controlled by the command signal in such a way that all doors and windows are closed. For example, this can prevent a cat from being injured on a tilted window.

In case that the external device is the smart home server, the room temperature and/or the lighting can be changed in dependency of the result of the evaluation, in particular the action-triggering result “undercooled”, “overheated”, “fall-asleep-event” and/or “wake-up-event”.

In one embodiment, the room temperature can be adjusted by the command signal in a targeted manner, in particular in dependency of the measured vital parameter, e.g. the body temperature. If the result is “undercooled”, the room temperature is automatically increased.

It may also be made possible for a person to take freshly baked rolls and/or fresh coffee from a corresponding kitchen device such as an oven, coffee maker or kitchen appliance when the person has reached the kitchen after getting up.

In particular, the action-triggering event is a predicted time of occurrence of an event, preferably the wake up time and/or fall asleep time (point in time). It can thus be made possible for an external device, in particular a household appliance (device), to carry out an action depending on the predicted time and thus save a particularly large amount of time for the user. If, for example, the organism is an infant, by means of predicting the wake up time, an external device for warming up a milk bottle or a kitchen appliance for preparing baby food can be activated so early that the milk bottle or baby food is ready (preparation finished) shortly before or at least at the same time or approximately at the same time as the infant wakes up. Parents can thus save time and sleep longer. Alternatively or in addition, the external device can be a notification device for one person or two persons, e.g. for one parent or both parents. In one embodiment, the system comprises an additional sensor and an additional control unit for both persons. On the basis of the respectively measured vital parameter, which is in particular transmitted to the evaluation unit in the form of a measurement value, the current physical condition of the persons can be determined.

In one embodiment, the evaluation unit conducts an evaluation, the result of which indicates the person who is suitable or most suitable among all persons to be notified on the basis of the measured vital parameters. In particular, a notification includes waking a person to be notified when being asleep. For example, a person is suitable for notification if the person is in a sleep phase above a threshold. This person then does not sleep deeply, so that waking up is comparatively less stressful for the person. For example, a person is more suitable than another person if the evaluation of the measured vital parameters shows that the person sleeps less deeply than the other person.

In one embodiment, the person to be notified is notified in such a way that only the notified person is awakened from sleep, but not another person directly next to him. This can be achieved, for example, by a vibrating alarm generator that can apply a vibrating alarm particularly quietly to a skin surface, for example. Overall, only a parent who is not deeply asleep can be awakened, for example, when it is determined that an infant wakes up or when a wake up time is predicted, in order to go to an external milk bottle and/or baby food preparation device, which has already been activated by the system, to take out the finished milk bottle or baby food portion, and to administer it to the infant. The sleeping time of the parents in total can thus be maximized.

When the sensor measures a vital parameter of an organism, it is particularly provided that the sensor transmits a corresponding sensor signal to a control unit connected to the sensor. Preferably, the sensor is connected to the control unit for transmitting the sensor signal or for data exchange with the control unit. In one embodiment, the sensor and the control unit are integrated in a transmitting device. In one embodiment, the control unit can conduct signal processing of the sensor signal, i.e. signal conversion and/or signal change. Preferably, the control unit can perform an analog-to-digital conversion and/or a signal change by an algorithm. A sensor signal is an particularly analog signal, whose voltage, current and/or frequency correlates with the measured vital parameter, i.e. its measured value. In one preferred embodiment, the measurement signal, which is generated on the basis of the sensor signal and provided to the evaluation unit, corresponds to the measured value of the vital parameter. In one embodiment, the sensor signal is transmitted by the control unit and/or the transmitting device to the evaluation unit only in the form of the measurement signal. In particular, the control unit then merely performs an analog-to-digital conversion from an analog sensor signal to a digital measurement signal. The measurement signal is preferably digital. In one embodiment, the control unit and/or the transmitting device have a data interface, in particular for data exchange with the evaluation unit. Preferably, the data interface is arranged for wireless data exchange. For example, the data interface is a WLAN interface, radio interface and/or Bluetooth interface. The measurement signal can thus be transmitted particularly reliably and wirelessly to a remote and/or mobile evaluation unit.

In one embodiment, a smartphone or tablet PC comprises the evaluation unit. The number of components for the user can thus be reduced and it can be achieved a particularly simple operation, for example via an app. In one alternative or supplementary embodiment, the external device comprises the evaluation unit. If the external device comprises the evaluation unit, the command signal is sent via a data line or cable. A less reliable wireless interface can then be avoided. In particular, the evaluation unit is provided by implementing a program code in an existing device, which is already there for other reasons.

In one embodiment, the control unit comprises the evaluation unit. The number of components for the user can thus be reduced. If the control unit comprises the evaluation unit, data can be exchanged between the control unit and the evaluation unit without a wireless interface, i.e. via a cable connection.

In one embodiment, the evaluation unit comprises a processor, a memory and/or a computer program code. In a embodiment, the control unit comprises a processor, a memory and/or a computer program code. Computer program code means instructions that can be stored on a memory. A processor, memory and/or computer program code may be configured to perform a multi-step method. Though method steps, it can be conducted signal processing, evaluation, generating a command signal, sending a command signal to the external device and/or carrying out an action.

An evaluation based on a measured vital parameter is generally performed with the aid of an algorithm. The measurement signal and/or the sensor signal can then be used as input variable or input variables for the algorithm. As an output variable, the algorithm outputs the result that, in particular, represents predefined states and/or certain state changes. In one embodiment, the result can also be a “zero event”, i.e. no predefined state or no specific state change was determined by the evaluation. The result is then not an action-triggering result. This means that no command signal is then generated for an external device or the command signal is then also an empty signal, which does not cause the external device to execute a defined action based on the command signal.

In one embodiment, the result can be “undercooled”, “overheated”, “deep sleep phase” and/or “sleep phase with low sleep depth”. The result then represents a predefined state of the organism. It is thus an action-triggering result.

In one embodiment, the result can represent a “wake-up-event”, i.e. a change from a sleep state to a wakeful state, or a “fall-asleep-event”, i.e. a change from a wakeful state to a sleep state. The result then represents a certain state change of the organism. It is thus an action-triggering result.

In one embodiment, the result is output in the form of a digital code, for example “0”, “1”, “2”, “3” or “4”.

In one embodiment, the command signal comprises an assignment to one of several actions stored in the external device. In one embodiment, the command signal comprises an assignment to an external device so that an action is only executed in the assigned external device on the basis of the command signal. Depending on the measured vital parameter, different actions can be triggered in a targeted manner on one or more external devices.

The action in an external device is preferably defined by a program that is stored in a memory, especially of the external device. The command signal then activates a stored program. Several programs can be stored.

Alternatively or in addition, the action itself can be specified by the command signal. The command signal then corresponds, for example, to a control signal with control commands for a controller of the external device that translates these control commands into action.

An external device is an independent and/or existing device of the user. The external device is located at any location and at any distance relative to the sensor, which can measure the vital parameter of the organism. In general, the external device does not include a sensor being arranged to measure the vital parameters of an organism and being intended this application.

In one embodiment, the sensor and a control unit are integrated in a transmitting device. The vital parameter of the person can thus be measured at any time and provided to the evaluation unit, especially wirelessly.

In one embodiment, a sensor signal of the sensor is converted by the control unit into a measurement signal which correlates with the measured vital parameter and is provided to the evaluation unit. A wireless transmission of the measurement to the evaluation unit can thus be made possible. Furthermore, signal processing can already take place in the transmitting device to relieve the evaluation unit.

In one embodiment, the transmitting device is provided and arranged to be worn on the body of the organism. To be worn on the body means a close carrying on the body or wearing directly on the body in such a way that at least one movement of the body in the region where the transmitting device is worn can be reliably recorded by a sensor. In particular, a fastening device for attaching the transmitting device to the body is provided.

In one embodiment, the transmitting device is integrated into a wristband, a footband, a headband, glasses, a hearing aid or a headphone. The integration into a wristband or footband allows a high wearing comfort while at the same time being close to the body surface. A headband allows measurement on the scalp. Glasses, a hearing aid and headphones allow a sensor to come into direct contact with the scalp without being unpleasantly perceived by the user.

In one embodiment, at least two separate receiving devices and/or sensors are provided on only one organism. A particularly precise evaluation of the body condition can thus be enabled.

In one embodiment, the sensor is a skin contact sensor for measuring electrical voltage fluctuations on a skin surface of the organism, especially on the head. The measurement signals for an electroencephalogram can thus be provided. When the state changes from the sleep state to the wakeful state, a measured frequency of the voltage fluctuations changes from alpha waves to beta waves. Conversely, a measured frequency of voltage fluctuations changes from the awake to the sleep state from beta waves to alpha waves when the state changes. Alpha waves are waves with a frequency range between 8 and 13 Hz. Beta wave refers to a wave with a frequency range between >13 and 30 Hz. State changes such as a wake-up-event and a fall-asleep-event as well as states such as a deep sleep phase and a sleep phase with low sleep depth can thus be reliably evaluated and output as a result.

In one embodiment, by means of a skin contact sensor, the body temperature can be measured alternatively or additionally. The body temperature is a vital parameter that correlates, among others, with the sleep/wake cycle. State changes such as a wake-up-event and a fall-asleep-event as well as states such as “hypothermia” and “overheating” can thus be evaluated and output as results. Preferably, a skin contact sensor has a skin-friendly contact surface. Health risks can thus be reduced.

In one embodiment, the evaluation unit is configured such that a comparison with a threshold value is carried out for the evaluation of the measured vital parameter. The sensor signal or the measuring signal are thus compared with a threshold value. In particular, before comparing the sensor signal or the measurement signal with the threshold value, a signal change can take place using a signal change algorithm of the evaluation unit in order to be able to carry out a particularly reliable evaluation.

In the embodiment with a skin contact sensor for measuring electrical voltage fluctuations on a skin surface of the organism, especially on the head, the preferred threshold value is 11 to 15 Hz, for example 13 Hz. In one embodiment, if the measurement signal is initially lower than the threshold value and then reaches the threshold value, a result is output that is assigned to the wake-up-event. The organism has thus woken up. In one embodiment, if the measurement signal is initially greater than the threshold value and then reaches the threshold value, a result that is assigned to the fall-asleep-event is output. The organism has thus fallen asleep.

In one embodiment, the sensor is a gyrometer. A gyrometer is used for example to measure a rotational movement. By measuring the rotational movement, a measured value of the activity of an organism can be determined which can be correlated with a state change, e.g. a wake-up-event. In particular, a change of direction of a rotational movement is recorded and/or measured per time interval of e.g. ten seconds. If, for example, at least six changes of direction take place in a ten-second period, this is an indication of a wakeful state. At the same time, there is a steep increase in the number of changes of direction within a period of, for example, ten minutes before waking up, with a particularly approximately constant gradient over time. The use of a gyrometer as a sensor enables a particularly reliable prediction of the wake-up time. In one embodiment, if the measurement signal is smaller than a threshold value, e.g. six changes of direction in a ten-second period, and then the threshold value is reached (coming from below), the result of the evaluation is a “wake-up event”. In an alternative or supplementary embodiment, if the measurement signal is greater than the threshold value mentioned above and the threshold value is then reached (coming from above), the result of the evaluation is “fall sleep event” (impact event). Alternatively or additionally, the sensor is a force sensor, a force transducer, a piezo sensor and/or a strain gauge. The organism is in particular an infant.

In particular, the at least one sensor can be a moisture sensor for detecting a wet diaper and/or sweat secretion, a motion sensor mat for activity measurement, an odour sensor, in particular for methane, a pulse meter, a blood pressure meter, a brain current sensor for EEG and/or ECG, an oxygen measurement sensor, in particular for determining the sleep phase, an MRI device, in particular for determining a wake-up time, a thermal imaging camera, a night vision camera in particular for determining characteristic motion sequences, a camera with color resolution in particular for assigning the skin color, a blood sugar level sensor, a CO2 measuring device for respiratory air, a pupil size measuring device, a blinking frequency measuring device in particular for predicting a fall asleep time and/or a respiratory frequency measuring device. In one embodiment, the sensor or sensors are attached to an organism's sleeping place, in or on a blanket and/or in or on a sleeping bag. One or more sensors can be used to detect body posture for example during sleep. The system thereby distinguishes between postures that are taken during hypothermia and postures that are not taken during hypothermia. Depending on this, for example, a heater is controlled in such a way that hypothermia is avoided. Conversely, in one embodiment of the present disclosure, determined postures during sleep are used to detect overheating and, depending on this, to control an air conditioning system in such a way that overheating is counteracted.

In particular, the vital parameters may be one, two or three of the following: body temperature, activity, pulse, blood oxygen content, blood sugar level, brain current, characteristic movements, characteristic postures, sweat secretion, CO2 respiratory air content, respiratory rate, pupil size and/or blink frequency.

In one embodiment, at least two sensors for different vital parameters are provided. A state change can thus be determined particularly reliably by considering two different vital parameters. For example, a body posture as well as sweat secretion when an organism is sleeping can be monitored by suitable sensors and, in dependency of that, an air conditioning system can be controlled to create a pleasant temperature climate for sleeping.

In one embodiment, an environmental information is also taken into account in the evaluation. In particular, the environmental information is the weather or a weather forecast, lunar phase calendar, a schedule for a bus, train, garbage collection and/or a robot vacuum cleaner. Preferably, the evaluation unit has an internet interface to connect to a weather service, a smart home server, e.g. with the schedule of the robot vacuum cleaner and/or a public schedule for bus, train and garbage collection. In one embodiment, one or more temperature sensors for recording a room temperature, a brightness sensor for recording a room brightness, a humidity sensor for recording a room humidity and/or a microphone for recording traffic noise, ambient noises or personal noises such as speeches or snoring are provided.

In one embodiment, the evaluation unit comprises a machine learning algorithm for the evaluation and/or determination of the command signal. A machine learning algorithm for the evaluation makes it possible to determine a physical condition or a state change particularly reliably on the basis of the measured vital parameter. A machine learning algorithm for determining the command signal enables to cause the external device to carry out a particularly suitable action among several stored actions, taking into account the result of the evaluation. Overall, by using a machine learning algorithm, the system can be adapted to the preferences and peculiarities of the organism and/or user.

A machine learning algorithm generally assigns an output variable to one or more input variables and usually outputs it. The output variable can be the result and/or the command signal. A machine learning algorithm is formed in particular by a program code or algorithm. In particular, a machine learning algorithm is generated by a modelling phase and a subsequent identification phase in order to finally be able to predict a point in time for the occurrence of a state change in an application phase. In particular, the modelling phase takes place at the manufacturer's site. The identification phase can take place at the manufacturer and/or at the end user. The application phase then takes place at the end user. For test purposes, the application phase can take place at the manufacturer. In the modelling phase, a mathematical model, i.e. a system of equations, is created to assign one or more input variables to an output variable. A correlation of one or more vital parameters with a condition or a state change is taken into account, i.e. reflected in the mathematical equation system. Preferably, in the model building phase, a dynamic model and/or differential equation system for the assignment of the output variable to an input variable or to a combination of input variables is created. In the model or differential equation system, the measurement signals of one or more defined sensors serve as the input variable or input variables and the result and/or the command signal as the output variable. In the identification phase, the machine learning algorithm is supplied with a plurality of value pairs, each with one input variable and one output variable or each with several input variables and one output variable. In this way, the machine learning algorithm is optimized and adapted to reality. In particular, constants are optimized in a differential equation system of the machine learning algorithm on the basis of the supplied value pairs. By providing a feedback device, an output variable can be supplied to the machine learning algorithm by the end user, which will be discussed in more detail later.

In the application phase, the machine learning algorithm is used to determine, select and/or assign the result and/or the command signal based on the evaluated measurement signals.

In one embodiment, a feedback unit is provided by which a user can give a feedback to the machine learning algorithm. By providing a feedback device, an output variable can be supplied to the machine learning algorithm by the user. In particular, the user can give feedback on the time of occurrence of an event such as a certain state change like “wake up” or a defined state such as “undercooled”. The machine learning algorithm is configured in such a way that the event of the feedback should result in or ideally include an action-triggering result. The machine learning algorithm can thus, for example, “learn” and consider for example typical wake-up times of a certain infant.

In one embodiment, the feedback device includes a button or switch to report feedback on the occurrence of a defined event. In one embodiment, the feedback device is implemented by an app for a smartphone or tablet PC in order to be able to enter event-specific feedbacks.

In one embodiment, the evaluation unit is configured in such a way that the command signal can trigger activation, opening and/or unlocking of the external device. In an alternative or supplementary embodiment, the evaluation unit is configured in such a way that the command signal can trigger deactivation, closing and/or locking. The action is therefore activation, opening, unlocking, deactivating, closing and/or locking. For example, a kitchen appliance can be activated to prepare food (a meal), a front door can be locked and/or a window can be closed.

In one embodiment, the evaluation unit is configured in such a way that the command signal can trigger a change of a setting of the external device. The action is thus to change a setting of the external device. The external device can thereby be adapted to the current physical condition of the organism. For example, the set target room temperature of an air conditioning system, which is controlled in particular by the smart home server, can be adapted to the body temperature of the organism. For example, a kitchen appliance (food processor) with automatically generated recipes or automatically suggested recipe recommendations can (be provided enabling to) adapt the recipes to the measured vital parameters of a person as the organism. For example, if the body fat content is too high, the fat content in the ingredients of a recipe is reduced and/or a recipe with a low fat content is suggested. For people suffering from diabetes, the blood sugar can be measured by the sensor and/or a recipe can be adapted to the current blood sugar.

In one embodiment, the external device is included in the system and/or the external device is a household appliance, in particular a kitchen appliance (food processor), an oven or a smart home server.

Carrying out an action related to a household appliance based on the evaluation of a measured vital parameter enables the household appliance to carry out an action that is adapted to the user's needs without the user himself having to act. A food can, for example, be prepared automatically and/or self-acting based on the measured vital parameter.

Household appliance means an electrically operated device for use in private households. A household appliance can be an electrical kitchen appliance or cleaning appliance (device), in particular with an interface to the Internet, a WLAN interface and/or a connection to a smart home server. A household appliance within the meaning of this disclosure also includes do-it-yourself appliances (tools) such as cordless screwdrivers or drills as well as garden appliances such as lawn mower robots. A cleaning appliance is, for example, a robot vacuum cleaner. A household appliance or device can also be a smart home server that can automate processes with the help of networked and remote-controlled devices, switches and sensors, thus enabling particularly high living quality, safety and energy efficiency. Preferably, the smart home server is connected to house installations, building equipment and household devices such as lamps, blinds, roller blind, doors, windows, heating, oven, stove, food processor, refrigerator, washing machine, vacuum cleaner, television and/or audio equipment.

Special advantages arise when the household appliance is a kitchen appliance, an oven or a smart home server. A kitchen appliance can then adapt displayed recipes automatically to the organism. The smart home server can automatically link up the operation of the air conditioner or air purifier as well as the timing of opening and/or closing a roller blind and/or door with a condition or state change of the organism. A baking oven can, for example, bake bread rolls close to waking up and getting up of a person or automatically deactivate itself for safety reasons when a person falls asleep.

In one embodiment, the external device and the corresponding action triggered by the command signal is at least one of the following examples: a smart home server for correspondingly switching on and/or off a light source, a parking heater for correspondingly heating up a motor vehicle, an oven for correspondingly food preparation, a kitchen appliance for correspondingly preparing a food, an oven for correspondingly switching off for safety reasons, a coffee machine, tea machine and/or bread baking machine for corresponding activation, a telephone call acceptance device for correspondingly accepting, rejecting and/or forwarding a telephone call, a robot vacuum cleaner or lawn mower robot for corresponding activation and/or route planning, a heating system and/or an air conditioning system for corresponding setting of the desired room temperature, automatically closable windows for corresponding closing and/or opening, an automatic entrance door lock for corresponding locking and/or unlocking.

A further aspect of the disclosure concerns the use of the system according to the previously described aspect of the disclosure to solve the problem described at the beginning, wherein the result of the evaluation is a falling-asleep event and/or waking-up event. Thus, when a person changes from sleep state to wakeful state, the result of the evaluation based on the measured vital parameter orderly indicates that a “wake-up event” or “fall asleep event” has occurred. For example, a front door can then be locked or unlocked by the command signal according to a stored program. The organism is in particular a person.

In one embodiment of the previously described use, the organism is an infant and/or toddler. Infants, who have to be breastfed especially by bottle, often wake up at night and scream or cry because of hunger. This usually leads to both parents waking up, one parent to get up, preparing a bottle of lukewarm milk and/or food, calming the infant or toddler, respectively, and administering the prepared food. Both the infant or toddler, respectively, and both parents are often kept awake at night several times for a longer period of time. By measuring the vital parameter in the infant or toddler, respectively, to evaluate the sleep course and activating a kitchen appliance at a correspondingly appropriate time to prepare food for the infant, time can be saved during providing food for the infant.

In one embodiment, a kitchen appliance can, for example, be equipped with baby food the evening before. In an alternative or supplementary configuration, a milk bottle preparation machine or tea machine can be equipped with milk powder, for example. The command signal can then, close to the wake-up event, cause automated preparation of the corresponding food, e.g. by warming up a milk bottle or mixing milk powder and tempered water and/or keeping it at a defined temperature until it is removed. The food can thus be administered immediately after the child and parents wake up and the preparation time can be saved or at least be reduced. A particularly valuable time saving for the food preparation at night and/or a reduced germ formation by a reduced period of keeping the food warm can be obtained in this way.

A further aspect of the present disclosure concerns a method, particularly according to the aspect of the disclosure described at the beginning, in which a sensor measures a vital parameter of an organism and an evaluation unit conducts an evaluation based on the measured vital parameter. The evaluation unit sends a command signal to an external device, in particular a household appliance, in dependency of a result of the evaluation. The external device carries out an action based on the command signal. The external device can thus carry out an action adapted to the needs of the organism without the organism itself having to take any action. An automatic control of an external device in particular for a pet or animal as the organism is thus made possible. Further embodiments and advantages, which analogously also refer to this method, are described in connection with the aspect of the disclosure described at the beginning.

Another aspect of the present disclosure concerns a computer program product. The computer program product comprises instructions which, when the program is executed by a computer, cause it to conduct the steps of the method according to the preceding aspect of the present disclosure. In particular, the computer is the evaluation unit. The features, embodiments and effects of the system for solving the problem described at the beginning also analogously refer to this computer program product.

In the following, embodiment examples of the disclosure are explained in more detail also using figures. Features of the embodiment examples and further alternative or supplementary embodiment described below can be combined individually or in a plurality thereof with the claimed objects. The claimed scope of protection is not limited to the embodiment examples.

### BRIEF DESCRIPTIONS OF THE DRAWINGS

It is shown:

FIG. 1: Schematic illustration of a system that, based on a measured vital parameter, can send a command signal to an external device that can carry out an action based on the command signal;

FIG. 2: Schematic illustration of the structure of a system that, based on a measured vital parameter, can send a command signal to an external device that can carry out an action based on the command signal;

FIG. 3: Schematic illustration of a diagram with the frequency of electrical voltage fluctuations measured on a skin surface over time;

FIG. 4: Schematic illustration of a diagram which shows the measurement signals from a measurement of an activity over time.

### DETAILED DESCRIPTION

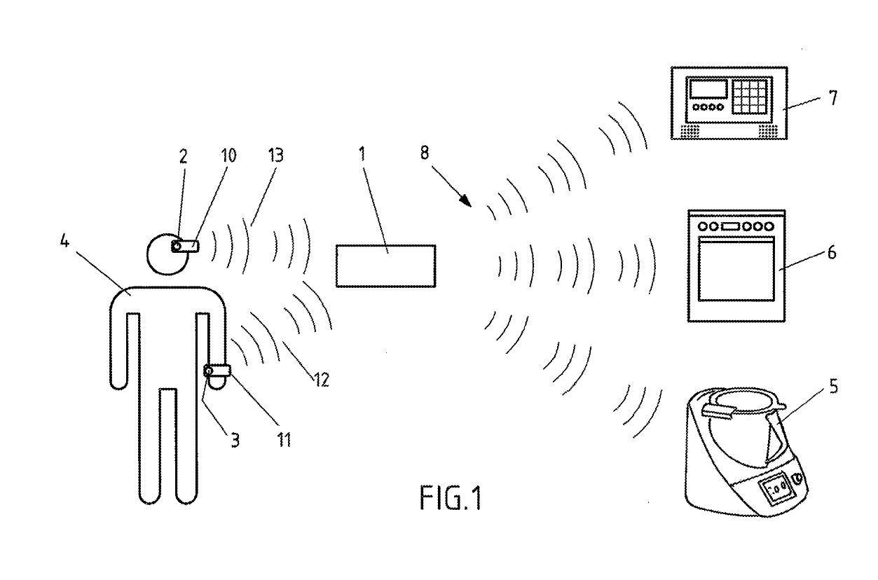

FIG. 1 shows an organism 4 carrying a transmitting device 10 with a skin contact sensor 2 on its head and/or another transmitting device 11 with another sensor, in particular a gyrometer 3, on its wrist. The transmitting device 10 can be integrated in glasses or a headband. Sticking or fastening it with a plaster can also be applied. The organism 4 is a human organism or a person, respectively, and can be an infant, a toddler, a man or a woman. In particular, the skin contact sensor 2 is used to measure an electrical voltage on the skin surface so that voltage fluctuations can be determined from the sensor signal. Alternatively or in addition, the skin contact sensor 2 is used to measure the body temperature. The other sensor or gyrometer 3, respectively, is integrated in a wristband in such a way that one movement of the wrist is detected by the other sensor or the gyrometer 3, respectively.

The at least one transmitting device 10, 11 transmits, preferentially wirelessly, the at least one measuring signal 12, 13 to an evaluation unit 1 for evaluation. Depending on a result of the evaluation, the evaluation unit 1 generates a command signal 8, which is sent wirelessly to at least one external device 5, 6, 7. In particular, a kitchen appliance 5, an oven 6 and/or a smart home server 7 are provided as external device as shown. Preferably, the command signal 8 comprises device assignment information and command information. The command information triggers the action to be carried out by a certain external device 5, 6, 7. The device assignment information indicates the addressed external device 5, 6, 7 for which the respective command information is provided. Preferably, a command signal 8 can comprise several sets of device assignment information and associated command information. Several external devices 5, 6, 7 can carry out an action in parallel using the command signal 8.

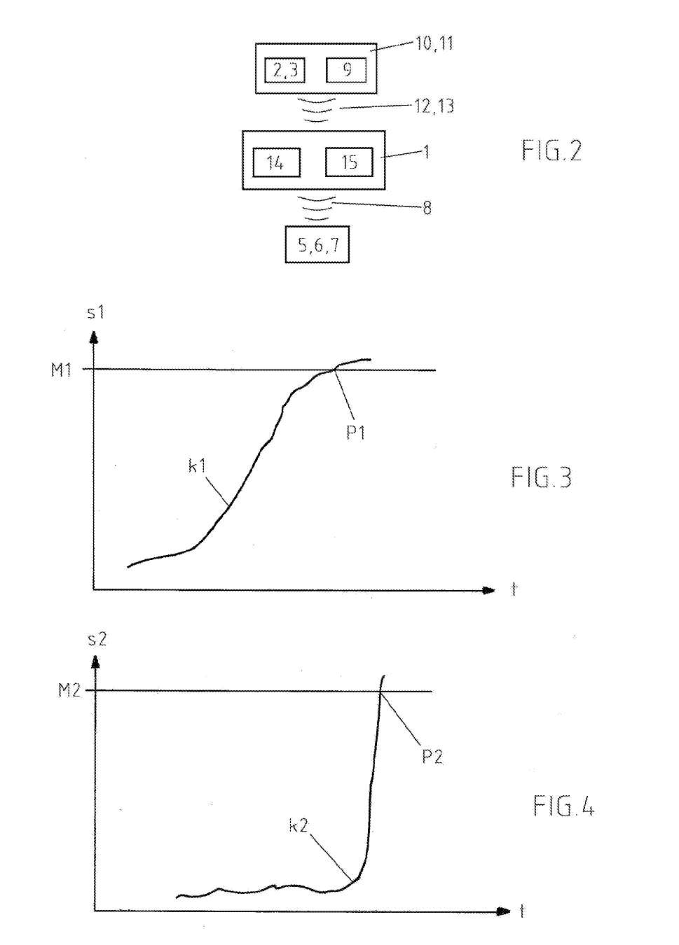

FIG. 2 shows a schematic structure of a system, in particular the one of FIG. 1. Each transmitting device 10, 11 comprises at least one sensor 2, 3 each. In one embodiment, two sensors 2, 3 can thus be integrated in one transmitting device 10, 11. Each transmitting device 10, 11 comprises one control unit 9.

The evaluation unit 1, which receives at least one measurement signal 12, 13 from the at least one transmitting device 10, 11 or the control unit 9 of the transmitting device 10, 11, comprises a processor 14 and a memory 15. In particular, the processor executes steps of a method which are stored in the memory 15 in the form of a program. Preferably, the program comprises a machine learning algorithm. The evaluation unit 1 generates a command signal 8 in dependency of a result of the evaluation and sends the command signal 8 to an external device 5, 6, 7 so that the external device 5, 6, 7 carries out an action based on the command signal 8, such as switching light on and/or off by the smart home server 7 or automatically preparing a food by the kitchen appliance 5 and/or by the oven 6.

FIG. 3 schematically illustrates a diagram resolved over time t in which a measurement curve k1 shows a vital parameter s1 with the measure value of a frequency of electrical voltage fluctuations on the skin surface at the head of the organism 4 measured by the skin contact sensor 2. In particular, the control unit 9 and/or the evaluation unit 1 comprise an algorithm for determining the frequency from a recorded course of the electrical voltage fluctuations, in particular from an electroencephalogram. During the state change from sleep state to wakeful state, i.e. during the “wake-up-event”, the frequency s1 changes from alpha waves to beta waves. Conversely, the frequency s1 changes from beta waves to alpha waves when the state change from the wakeful state to the sleep state, i.e. during the “fall-asleep-event”, occurs. A threshold value M1 is used particularly at a frequency of 12, 13 or 14 Hz. The evaluation includes a comparison of the measurement signal 11 or the measurement curve k1 with the threshold value M1. If the measurement signal is below the threshold value M1, the result is “sleep state”. If the measurement signal is above the threshold value M1, “wakeful state” is the result. If the M1 threshold is exceeded, “wake-up event” is the result. If the value falls below the M1 threshold, “Sleep event” is the result. In FIG. 3, such an exceeding occurs at the intersection P1 of trace k1 with the threshold value M1.

In one embodiment, an intersection point with a threshold value is predicted by extrapolating the measurement curve from measurement signals 11, 12 on the basis of a measurement curve. As a result, the predicted wake-up time and/or fall asleep time can be output. A particularly large saving of time can thus be achieved. In particular, this embodiment concerns the example in FIG. 3 with the measurement curve k1, the threshold value M1 and the intersection point P1. Alternatively or additionally, this embodiment particularly concerns the embodiment of FIG. 4 with the measurement curve k2, the threshold value M2 and the intersection P2.

FIG. 4 schematically illustrates another example of a diagram in which a measurement curve k2 represents a vital parameter s2 with the measured value of an activity over time t. In particular, the measurement curve corresponds to the measurement signals determined based on the sensor signals of the gyrometer 3, preferably on the wrist of the organism 4. The measure of activity corresponds to the number of changes of direction within a defined period of time, e.g. ten seconds. If a threshold value M2 is exceeded, e.g. six changes of direction within a period of ten seconds, the “wake-up event” is the result of the evaluation. In FIG. 4, such an exceedance occurs at intersection P2 of the trace k2 with the threshold value M2.

As described above, the external device 5, 6, 7 is in one embodiment a household appliance, namely a kitchen appliance 5 or a robot vacuum cleaner. If the household appliance is a robot vacuum cleaner, a schedule of the robot vacuum cleaner can be additionally taken into account in the evaluation. If the household appliance is a kitchen appliance 5, the action can be an automatic provision of an automatically generated recipe, an automatically suggested recipe recommendation and/or the display of a recipe by the kitchen appliance (5).

If the evaluation unit 1 determines a predicted time for the occurrence of an event on the basis of the measured vital parameter, in particular on the basis of the intersection of a measurement curve from measurement signals with a threshold value by extrapolation of the measurement curve, the following embodiments are enables. In one embodiment, the evaluation unit 1 sends the command signal for carrying out an action to a kitchen appliance or a robot vacuum cleaner at a defined time interval, i.e. time distance, before the predicted point in time. The time of completion of the action can thus be determined relative to the predicted time.

In one embodiment, the action is a deactivation, in particular an immediate deactivation, of a selection of household appliances or of all household appliances covered by the system to which the evaluation unit 1 can send command signal 8. In this way, the complexity of the control can be minimized and, at the same time, great time savings and user comfort can be achieved. For example, the household appliances are deactivated in time for falling asleep, so that noise emissions can be reduced and electricity saved by means of a very simple control. The defined time interval mentioned above can also support falling asleep by deactivating household appliances at the defined time interval before the predicted time of falling asleep.

Preferably, the cleaning appliance is a robot vacuum cleaner. In particular, a schedule for the robot vacuum cleaner is provided. The schedule preferably includes a route and/or a timetable for cleaning. For example, the timetable stipulates that the robot vacuum cleaner must regularly travel the route in order to clean the floor of living areas.

In one embodiment, the evaluation unit 1 is configured such that, in dependency of a result of the evaluation of a measured vital parameter, the evaluation unit sends a command signal 8 to the robot vacuum cleaner or a control system for administrating the schedule of the robot vacuum cleaner, wherein the command signal 8 causes the schedule, i.e. the route and/or the timetable, to be changed in dependency of a result of the evaluation of the vital parameter. The route can thereby be changed such that the bedroom for example is widely bypassed if the organism is close to the fall-asleep-event or wake-up-event. Alternatively or additionally, the schedule can be changed such that the robot vacuum cleaner stops cleaning close to the fall-asleep-event or wake-up-event (especially at the defined time interval from the predicted time of the fall-asleep-event or wake-up-event) or stops cleaning temporarily and continues cleaning at a later time.

A kitchen appliance 5 has at least the three functions of heating, chopping and blending a food. Preferably, the kitchen appliance can access stored recipes for a variety of foods. Preferably, a recipe can be displayed on the kitchen appliance via an interactive display, e.g. touch screen display, and processed by the user step by step. In one embodiment, the kitchen appliance can process a recipe completely self-acting and thus automatically prepare a food (dish).

In one embodiment, the evaluation unit 1 is configured in such a way that, in dependency of the result of the evaluation of a measured vital parameter, the evaluation unit sends a command signal 8 to the kitchen appliance. This command signal 8 causes that, in dependency of a result of the evaluation of the vital parameter, suggestions for recipe changes or recipes that have already been adapted accordingly are displayed, in particular via the display of the kitchen appliance. In this way, the user can take his body condition with special care and awareness thereof into account when preparing food with the help of the kitchen appliance and with the support of the system. This allows a significant time saving and a significant increase in user comfort.

In the case of automatic food preparation, it can be provided that the underlying recipe can be changed directly. This also saves the user the time of adapting his food to his physical condition, e.g. in the case of obesity or diabetes.

1. A system comprising
an evaluation unit and a sensor, wherein the sensor is configured to measure a vital parameter of an organism and the evaluation unit is configured to conduct an evaluation based on the measured vital parameter,
wherein the evaluation unit is configured such that the evaluation unit can send a command signal to an external device in dependency of a result of the evaluation, so that the external device carries out an action based on the command signal.

2. The system of claim 1, wherein the external device is a household appliance provided by a kitchen appliance or a robot vacuum cleaner.

3. The system of claim 2, wherein the sensor and a control unit are integrated in a transmitting device and a sensor signal of the sensor is converted by the control unit into a measurement signal which correlates with the measured vital parameter and is provided to the evaluation unit.

4. The system of claim 3, wherein the transmitting device is sized and configured to be worn on the body of the organism.

5. The system of claim 1, wherein the sensor is a skin contact sensor configured to measure electrical voltage fluctuations on a skin surface of the organism.

6. The system of claim 1, wherein the evaluation unit is configured such that a comparison with a threshold value (M1, M2) is carried out for the evaluation of the measured vital parameter.

7. The system of claim 1, wherein two sensors for different vital parameters are included in the system.

8. The system of claim 1, wherein the evaluation unit comprises a machine learning algorithm for the evaluation or determination of the command signal.

9. The system of claim 8, wherein a feedback unit is provided by which a user can give a feedback to the machine learning algorithm.

10. The system of claim 1, wherein the evaluation unit is configured such that the command signal can trigger an activation and/or a deactivation of the external device.

11. The system of claim 1, wherein the evaluation unit is configured such that the command signal can trigger a change of a setting of the external device.

12. The system of claim 1, wherein the external device is included in the system and is one of a kitchen appliance, an oven or a smart home server.

13. (canceled)

14. (canceled)

15. (canceled)

16. (canceled)

17. The system of claim 2, wherein the household appliance is a robot vacuum cleaner and a schedule of the robot vacuum cleaner is additionally taken into account by the evaluation unit in the evaluation.

18. The system of claim 2, wherein the household appliance is a kitchen appliance, and the kitchen appliance is configured to perform at least one of the following as the action:
an automatic provision of an automatically generated recipe, an automatically suggested recipe recommendation and display of a recipe by the kitchen appliance.

19. The system of claim 4, wherein the transmitting device is integrated in a wristband, a footband, a headband, glasses, a hearing aid or a headphone.

20. A method comprising the steps of
measuring a vital parameter of an organism via a sensor,
conducting an evaluation based on the measured vital parameter via an evaluation unit,
sending a command signal from the evaluation unit to an external device depending upon the result of the evaluation, and
carrying out an action based on the command signal by an external device.

21. The method of claim 20, wherein the result of the evaluation is a falling-asleep event or waking-up event.

22. The method of claim 21, wherein the organism is an infant.

23. A computer readable medium comprising instructions which, when the instructions are executed by a processor, cause the processor to perform a method comprising
evaluating a measurement signal from a sensor, the measurement signal associated with a vital parameter of an organism,
conducting an evaluation based on the measured vital parameter, and
sending a command signal from the evaluation unit to an external device depending upon the result of the evaluation.
